(19) United States
(12) Patent Application Publication
(10) Pub. No.: US 2025/0325780 A1
(43) Pub. Date: Oct. 23, 2025

(54) PSYCHOTHERAPY VIRTUAL REALITY SYSTEMS AND METHODS OF USE

(71) Applicant: Jacques Jospitre, New York, NY (US)

(72) Inventor: Jacques Jospitre, New York, NY (US)

(21) Appl. No.: 19/184,811

(22) Filed: Apr. 21, 2025

Related U.S. Application Data
(60) Provisional application No. 63/636,546, filed on Apr. 19, 2024.

(51) Int. Cl. A61M 21/00; G06F 3/01

(52) U.S. Cl. CPC A61M 21/00; G06F 3/015; A61M 2021/005; A61M 2230/00

(57) ABSTRACT

A psychotherapy virtual reality system configured to be used in the context of a psychotherapy session by a controlling user and a participating user is provided. The psychotherapy virtual reality system includes a virtual reality device configured to provide a virtual reality environment, a database including one or more data elements derived from information associated with the participating user, and a processor and memory. Each data element is representative of the information. The processor is configured to, in a psychotherapy session: select, by the controlling user, a data element from the database to be used to construct a scenario in the virtual reality environment; present, in the virtual reality environment provided by the virtual reality device, the scenario including a virtual reality representation of the selected data element to the participating user; and control, by the controlling user, the virtual reality representation of the selected data element.

### CROSS-REFERENCE TO RELATED APPLICATIONS

This application claims priority to and the benefit of U.S. Provisional Patent Application No. 63/636,546 filed on Apr. 19, 2024, the entire disclosure of which is hereby incorporated by reference.

### BACKGROUND OF THE INVENTION

The present subject matter relates generally to a virtual reality system configured to depict a patient's environment in order to provide therapy treatment. More specifically, the present invention relates to a psychotherapy virtual reality (VR) system configured to present a virtual reality environment tailored to the patient's emotional health through which the patient and/or therapist interact in order to effectuate therapy treatment.

VR systems are digitally rendered environments in which users immerse themselves in a virtual experience. These virtual environments can be modeled after real or imaginary environments, and can include elements that are based in reality as well as elements that have no basis in reality.

Current technology allows users to explore these environments using devices such as a head-mounted display (HMD). HMDs display a virtual environment in front of the user's eyes. The HMDs can take a variety of forms, such as glasses, goggles, helmets etc. Alternatively, non-immersive or semi-immersive VR experiences can be provided through a computer or gaming console.

VR systems are most commonly used for gaming. Secondary but also common uses are exercise, education, and job training.

There is a need for a virtual reality system that allows the user to safely explore and interact with an environment specific to the user's mindset in order to provide therapy treatment, as described herein.

### BRIEF SUMMARY OF THE INVENTION

To meet the needs described above and others, the present disclosure provides a psychotherapy VR system that presents a virtual environment tailored to a patient's emotional health and allows the patient's therapist and the patient to interact within the virtual environment to accomplish treatment goals. While the term “therapist” is used throughout this application, the psychotherapy VR system may be used by any mental health professional, such as but not limited to a psychologist, a psychiatrist, a psychoanalyst, a counselor, a virtual agent, or a virtual psychologist. Similarly, the term “patient” is used throughout the application, while the psychotherapy VR system may be used by a patient, a client, or any individual receiving treatment or working with a mental health professional.

In one embodiment, a psychotherapy VR system of the present application is configured to be used in the context of a psychotherapy session. The psychotherapy VR system includes a VR device, such as an HMD, a database, and a processor or controller in communication with the VR device and the database. Both the therapist and the patient engage with the psychotherapy VR system. Throughout the specification, the term “controlling user” refers to the therapist or other mental health professional, and the term “participating user” refers to the patient, client, or individual receiving treatment from the mental health professional. The processor or controller includes a memory for storing program instructions to implement the methods described herein.

The VR device is configured to provide a VR environment to the participating user during a psychotherapy session through which the participating user and the controlling user can engage or interact. The controlling user determines the scenario that is presented to the participating user through the VR environment. For example, the controlling user defines the participating user's VR environment by selecting features and components of the scenario that align with the controlling user's treatment plan for the patient.

The database includes data elements that are derived from information associated with the participating user and may be represented by a virtual reality representation in the VR environment presented to the participating user. Each data element is representative of the information associated with the participating user. The information may include elements of the participating user's past, such as stories from childhood, traumatic events from the user's past, or details of the user's experiences and environments.

In one example, the information may include stories from the participating user about being bullied in the fifth grade on the playground of the participating user's elementary school by a bullying classmate. The stories may describe the bullying classmate physically attacking the participating user in a first fight. In this example, data elements derived from the information include the bully classmate, the elementary school playground, and first fighting.

Generally, the data elements may include one or more of a person, an event, a scenario, a series of events, a location, a place, a sound, or a scent. In another example, the participating user may describe a car accident that caused severe injuries to a close friend in the high school parking lot. The data elements based on this information may include the car accident (event and/or sound) occurring in the school parking lot (scenario), and the close friend (person). In a further example, the participating user may describe a fear of heights experienced while hiking. The data elements based on this information may include, among other things, the Grand Canyon (place), hiking (activity), and heights in general (scenario).

As noted above, the psychotherapy VR system enables the controlling user to construct a scenario in the VR environment through which the participating user is treated during a psychotherapy session. For example, the controlling user selects one or more data elements from the database to be featured in a scenario of the VR environment. The controlling user may select a data element as part of the participating user's treatment during the psychotherapy session and uses the selected data element to construct the scenario for the VR environment. The constructed scenario includes a virtual reality representation of the selected data element.

For example, where the controlling user decides to focus a psychotherapy session on addressing the participating user's fear of heights, the controlling user may select the data element of heights and/or the Grand Canyon. The VR representation of the data element of heights may be hiking on a mountain, riding a Ferris wheel, riding a ski lift, or sitting in the nosebleed seats in an amphitheater.

In another example, the controlling user may select the data element of the participating user's bullying classmate from fifth grade for a session focused on addressing the participating user's emotional stress related to bullying from childhood. The VR representation of the bully may include an avatar of the bully, an image of the bully from the participating user's yearbook from fifth grade, a faceless anime figure with bullying characteristics, or a deepfake representation of the bullying classmate.

Once the VR environment is presented to the participating user through the VR device, the participating user may engage with the scenario, including the components of the scenario such as the VR representation of the data element. The VR representation of the selected data element in the VR environment is controlled by the controlling user. In the heights example described above, the participating user may interact with the VR environment by skiing down a mountain, experiencing the rush and thrill of skiing, and then board the ski lift to return to the top of the mountain. The controlling user modifies the selected data element by selecting and modifying the terrain of the mountain, the location of the ski lift at the bottom thereof, the windiness of the environment when boarding the ski lift, and distractions from other skiers when on the ski lift. In the bullying example, the controlling user controls the presentation and dialogue of the bully, and the participating user may speak to and interact with the bullying classmate, standing up to the bully and advocating for himself.

In one embodiment of the present invention, a psychotherapy virtual reality system is configured to be used in the context of a psychotherapy session by a controlling user and a participating user. The psychotherapy virtual reality system includes a virtual reality device configured to provide a virtual reality environment, a database including one or more data elements derived from information associated with the participating user and provided by one of the controlling user and the participating user, a processor in communication with the virtual reality device and the database, and a memory. Each data element is representative of the information. The memory is in communication with the processor and stores program instructions that, when executed by the processor, cause the processor to, in a psychotherapy session, select, by the controlling user, a data element from the database to be used to construct a scenario in the virtual reality environment; present, in the virtual reality environment provided by the virtual reality device, the scenario including a virtual reality representation of the selected data element to the participating user; and control, by the controlling user, the virtual reality representation of the selected data element.

In some embodiments, each data element comprises one of a person, an event, an activity, a series of events, a location, a place, a sound, a scent, or a word or phrase. In other embodiments, the scenario includes a plurality of virtual reality representations. For example, the data elements may include a person, a place, and a phrase.

In a further embodiment, the processor is further configured to: construct a dataset of the one or more data elements based on the information; and store, via the database, the dataset. The step of constructing the dataset may include identifying, by the controlling user, an element disclosed during a conversation between the controlling user and the participating user, wherein each identified element corresponds to a data element. Alternatively or additionally, the step of constructing the dataset comprises inputting one or more elements associated with the participating user, wherein each inputted element corresponds to a data element.

In a still further embodiment, the database may further include information derived from one or more external data sources, such as satellite imagery from transportation departments.

In other embodiments, the processor is further configured to capture responsive data related to the participating user. For example, the responsive data comprises physiological data. The psychotherapy virtual reality system may further include one or more biological sensors configured to be worn by the participating user, and the step of capturing the responsive data may include receiving, from the biological sensors, the physiological data.

In other embodiments, the processor is configured to repeatedly present the virtual reality representation of the selected data element to the participating user during a plurality of psychotherapy sessions; repeatedly capture the responsive data during the plurality of psychotherapy sessions; and determine a data trend based on the captured responsive data. The data trend indicates an improvement, no change, or a decline.

In another embodiment, each data element is associated with a category. For example, each data element is associated into one or more of the following categories: anger, disgust, sadness, fear, shame, joy, pride, anxiety, excitement, or anticipation. Categories may group data elements 112 based on other commonalities such as priority level, timeframes (childhood, adulthood, older age), or any other basis as desired. The processor may be further configured to: present, to the controlling user, one or more additional data elements, each additional data element being associated with the same category as the selected data element; select, by the controlling user, an additional data element from the one or more additional data elements; and present, in the virtual reality environment provided by the virtual reality device, a virtual reality representation of the selected additional data element to the participating user.

An object of the invention is to provide a safe, virtual environment through which a therapist can lead a patient through emotional development and growth.

Another object of the invention is to provide a virtual environment in which the patient can interact with virtual elements that have emotional meaning specific to the patient.

Additional objects, advantages, and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a schematic of a psychotherapy virtual environment system of the present application.

FIG. 2 is an exemplary flowchart of a method performed by the psychotherapy virtual environment system of FIG. 1.

### DETAILED DESCRIPTION OF THE INVENTION

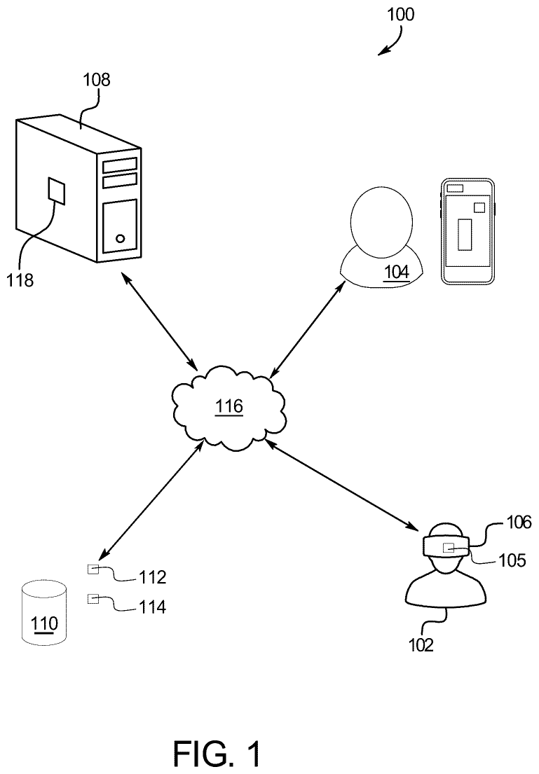

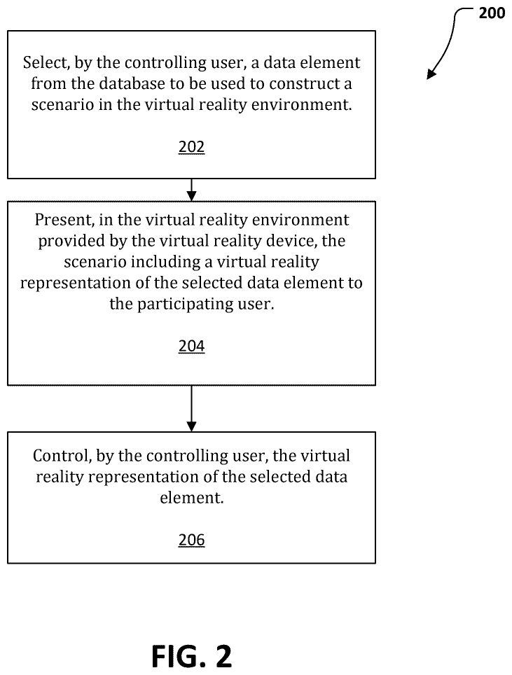

FIGS. 1 and 2 illustrate an example of a psychotherapy virtual reality (VR) system 100 of the present application. The psychotherapy VR system 100 is configured for use by a patient or client, referred to as a participating user, 102 and a therapist or mental health professional, referred to as a controlling user, 104. The psychotherapy VR system 100 presents a virtual environment 105 tailored to a patient's emotional health and allows the patient's therapist and patient to interact within the virtual environment 105 to accomplish treatment goals.

In one embodiment, a psychotherapy VR system 100 of the present application is configured to be used in the context of a psychotherapy session. The psychotherapy VR system includes a VR device 106, such as an HMD, a processor or controller 108, and a database 110. The processor 108 is in communication with the VR device 106 and the database 110. The devices 106, 108, 110 of the psychotherapy VR system 100 may communicate over wireless or wired internet 116.

The processor 108 includes a memory 118 in communication with the processor 108, the memory 118 storing program instructions that, when executed by the processor, cause the processor 108 to display the virtual environment 105 and undertake additional steps discussed herein. While the virtual reality device 106 is a head-mounted device in FIG. 1, the virtual reality device 106 may be any type of device displaying a virtual environment 105 to the participating user 102.

The psychotherapy VR system 100 displays the VR environment 105 including virtual reality representations 114 of one or more data elements 112 associated with the participating user 102. The controlling user 104 provides treatment to the participating user 102 on emotional development through the virtual environment 105 by controlling the virtual reality representations 114 of the data elements 112. The controlling user 104 may pursue treatment to address past or current events as well as to set goals and intentions and other life coaching purposes.

More specifically, the VR device 106 is configured to provide a VR environment 105 to the participating user 102 during a psychotherapy session through which the participating user 102 and the controlling user 104 can engage or interact. The controlling user 104 determines the scenario that is presented to the participating user 102 through the VR environment 105. The controlling user 104 defines the participating user's VR environment 105 by selecting features and components of the scenario that align with the controlling user's treatment plan for the participating user 102.

At least one of the selected features and components of the scenario includes a data element 112 and the related virtual representation 114 thereof stored on the database 110. The data elements 112 are derived from information associated with the participating user 102. For example, each data element 112 is representative of the information associated with the participating user 102.

In one embodiment, the information from which the one or more data elements 112 is derived is details and elements of the participating user's past, such as stories from childhood, traumatic events from the user's past, or details of the user's experiences and environments. The information may include, but is not limited to, factual data about the participating user's experiences, the participating user's reactions to events or situations, historical information or current events related to the participating user's past.

The information may be provided or collected through a variety of methods. In one embodiment, the information includes statements, comments, or stories disclosed during conversation between the participating user 102 and the controlling user 104. In other embodiments, the information includes responses or answers from an intake survey prepared by the controlling user 104 and completed by the participating user 102. In a still further embodiment, the information includes answers or responses from an intake survey prepared by the controlling user 104 and completed by the controlling user 104 based on details provided by the participating user 102 during an initial psychotherapy session. The data elements 112 stored on the database 110 may be collected through an iterative process over a period of time. The data elements 112 can be provided directly to the database 110 from the participating user 102 and/or the controlling user 104 through one or more user devices, such as a mobile device. The controlling user 104 may update the database 110 regularly based on therapy sessions, conversations, and other input means. The psychotherapy VR system 100 may also utilize machine learning to deduce an emotional meaning from data elements 112, including feedback from the controlling user 102 during interactions with the psychotherapy VR system 100.

The data elements 112 derived from the information may include a person, an event, an activity, a series of events, a location, a place, a sound, or a scent. The data elements 112 may also include images, music, journal entries, and dream journals. In one example, the information associated with the participating user 102 may include a description of the participating user 102 getting bullied in the fifth grade on the playground of the participating user's elementary school by a classmate who physically attacked the participating user. Data elements 112 derived from the information may include the bullying classmate, the elementary school playground, first fighting generally, and words or phrases spoken by the bullying classmate.

In another example, the participating user 102 may describe a car accident that caused severe injuries to a close friend in the participating user's high school parking lot. The data elements 112 based on this information may include the car accident (event or sound) occurring in the school parking lot (scenario), and the close friend (person). In a further example, the participating user may describe a fear of heights experienced while hiking in the Grand Canyon. The data elements 112 based on this information may include the Grand Canyon (place) and fear of heights or heights in general (scenario).

In a still further example, the participating user 102 may describe a fear of plane rides and potential plane crashes. The data elements 112 based on this information may include planes (place), flying in planes generally (activity), and a plane crashing generally (activity). This example demonstrates that the data elements 112 are not limited to past or historical events of the participating user 102 and may include potential events or general thoughts or concerns.

In other embodiments, the database 110 may also store emotional meanings of the data elements 112. The emotional meanings of the data elements 112 may comprise a dataset of meanings for specific aspects of the patient user input. For example, the data set may include a sudden, painful sadness associated with the sound or imaging of a car accident, indicative of losing a loved one, or a hostile feeling associated with the image of the patient's bully.

Referring back to FIG. 1, the psychotherapy VR system 100 enables the controlling user 104 to construct a scenario in the VR environment 105 through which the participating user 102 is treated during a psychotherapy session. In one example use of the psychotherapy VR system, the controlling user 104 may pursue treatment for a participating user 102 focused the user's fear of heights. The controlling user 104 selects a data element 112 of heights from the database 110 to be featured in a scenario of the VR environment 105. The scenario presented in the VR environment 105 by the VR device 106 to the participating user 102 includes a virtual reality representation 114 of the selected data element 112.

In this example, the controlling user 104 may select the data element 112 of heights for a session focused on addressing the participating user's fear of heights. The VR representation 114 of the data element 112 may be hiking on a mountain, riding a Ferris wheel, sitting on a ski lift as it moves up a mountain, or sitting in the nosebleed seats in an amphitheater.

In another example, the controlling user may select the data element 112 of the participating user's bully from fifth grade for a session focused on addressing the participating user's emotional stress related to bullying from childhood. The VR representation 114 of the bully may include an avatar of the bully, an image of the bully from the participating user's yearbook from fifth grade, or a faceless anime figure with bullying characteristics. In a further example, the psychotherapy VR system 100 generates a deep fake of the bully and incorporates the deep fake into the virtual environment displayed to the patient 102 on the virtual reality device 106. The processor 108 receives input from the controlling user 104 to control and manipulate the deep fake during interactions with the participating user 102 in the virtual environment.

In a still further example, the participating user 102 may describe setting a goal of developing a side project into a business, developing a business to reach specific profitability goals and/or scaling to specific levels, setting intentions for long term goals, and/or identifying, prioritizing, and achieving long term life goals. The data elements 112 may include the business goals or long-term goals (activities). The controlling user 104 may select data elements of 112 related to these specific goals or to more general overarching goals.

With the VR environment presented to the participating user 102 through the VR device 106, the participating user 102 engages with the scenario, including the components of the scenario such as the VR representation 114 of the data element 112. The VR representation 114 of the selected data element 112 in the VR environment 105 is controlled by the controlling user 104. In the heights example, the participating user 102 may interact with the VR environment 105 by skiing down a virtual mountain and physically experiencing the rush and thrill of skiing, and then board the ski lift to return to the top of the mountain. The controlling user 104 modifies the VR environment 105 and/or the VR representation 114 of the selected data element 112 by selecting the terrain of the mountain, the location of the ski lift at the bottom thereof, the security features on the ski lift, the presence of others on the ski lift with the participating user 102, and whether others on the ski lift remain calm and still or rowdy and cause the ski lift to swing. By varying the scenario through which the VR representation 114 of the selected data element 112 is presented, the controlling user 104 is able to observe and talk through the participating user's experience with those variables in real time.

In the bullying example, the controlling user 104 may control the VR representation 114 of the selected data element 112 by controlling the appearance, movements, and dialogue of the bully in the VR environment 105. The participating user engages with the scenario in the VR environment 105 by speaking to the bully, standing up to the bully, and advocating for himself. The controlling user 104 observes and discusses the participating user's experience with those variables in real time.

In the long term goals example, the controlling user 104 may control the VR representation 114 of the data elements 112 related to goal setting activities by engaging with the participating user 102 with visualization practices or other similar practices in the VR environment 105.

Referring to FIG. 2, the psychotherapy virtual reality system 100 is configured to be used in the context of a psychotherapy session by a controlling user and a participating user. The system 100 performs a method 200 including the steps of selecting, by the controlling user, a data element from the database to be used to construct a scenario in the virtual reality environment in step 202; presenting, in the virtual reality environment provided by the virtual reality device, the scenario including a virtual reality representation of the selected data element to the participating user in step 204; and controlling, by the controlling user, the virtual reality representation of the selected data element in step 204. The method 200 may include additional steps outlined herein.

In some examples, the scenario is constructed from a plurality of data elements 112. Referring to the bullying example, the data elements 112 derived from the information may include the bullying classmate (a person), the elementary school playground (a place), first fighting (an event or activity), and threatening phrases that the bully asserted during the incident (a phrase). The scenario presented in the VR environment 105 may include structures of the elementary school playground and an avatar of the bullying classmate articulating the threatening phases. This scenario, which includes a plurality of VR representations 114 of data elements 112, is based on only data elements 112 derived from information from the participating user 102.

In one embodiment, the database 110 may store a dataset of the one or more data elements 112 thereon. For example, the dataset may be constructed by identifying, by the controlling user 104, an element disclosed during a conversation between the controlling user 104 and the participating user 102 that corresponds to a data element 112. During a psychotherapy session, the participating user 102 may describe the user's experiences in fifth grade generally, including details about friendships, siblings, teachers, and the like. The participating user 102 describes a bullying incident as part of the overall discussion, describing his reactions to threatening phrases from the bully and first fighting. The controlling user 104 may identify the elements of the bully, the threatening phrases, and first fighting within the information from the participating user 102 as data elements 112 for storing on the database 110.

In some embodiments, the psychotherapy VR system 100 is configured to monitor the conversation between the participating user 102 and the controlling user 104 and automatically generates a list of elements based on the number of times an element is mentioned or based on words used in the description, such as “threatening” or “bullying.” The controlling user 104 may identify specific elements as data elements 112 for storage in the database. In other embodiments, the controlling user 104 inputs one or more elements associated with the participating user 102 into the database 110 through a user interface presented on a device such as a mobile device or a computer, each inputted element corresponding to a data element 112.

In some embodiments, the database 110 may further include information derived from one or more external data sources. For example, the database 110 may store the participating user's elementary school playground as a data element 112 as well as satellite images of elementary school playground collected from external data sources, such as imagery from a local transportation department.

In some embodiments, the psychotherapy VR system 100 is configured to capture responsive data related to the participating user 102. The responsive data may include but is not limited to physiological data, observations of the participating user 102 engaging with the scenario in the VR environment in real time, and feedback from the participating user 102 collected over time.

For example, in one embodiment, the responsive data is physiological data, and the psychotherapy VR system 100 includes one or more biological sensors worn by the participating user 102 to detect the physiological data. Capturing the responsive data includes receiving the physiological data from the one or more biological sensors.

In another embodiment, the responsive data is input observation data provided by the controlling user 104 and/or the participating user 102. The input observation data may include comments, characterizations, ratings, or other descriptions of the participating user's response to the VR representations 114 of the selected data elements 114.

The psychotherapy VR system 100 may collect and track responsive data over a period of time. For example, the system 100 may repeatedly present the virtual reality representation(s) of the selected data element(s) to the participating user 102 during a plurality of psychotherapy sessions and repeatedly capture the responsive data during the plurality of psychotherapy sessions. The system 100 may determine a data trend based on the captured responsive data. The data trend may indicate an improvement, no change, or a decline. For example, the system 100 may capture responsive data by monitoring the participating user's heartrate through a heartrate sensor. The system 100 determines that the participating user's heartrate increases in response to a VR representation 114 of a data element 112, such as a bully or heights. The system 100 may detect that the user's initial responsive heartrate increases when presented with the VR representation 114 of the selected data element 112 and that the increase or jump in heartrate when initially presented with the VR representation 114 minimizes over time, indicating that the participating user 102 is less affected by the data element 112 as time progresses. The system 100 may determine a data trend of an improvement based on this responsive data. In other embodiments, the data trend may indicate no change, a decline, or another interpretation. In some further example embodiments, the system 100 may output the data trend.

In still further embodiments, each data element 112 may be associated with a category. The categories may correspond to emotional stages such as anger, disgust, sadness, fear, shame, joy, pride, anxiety, excitement, or anticipation. In one embodiment, the system 100 may enable the controlling user 104 to select a category of data elements 112 prior to selecting the data element 112. For example, the controlling user 104 wishes to pursue treatment on the participating user 102 related to the user's fears. The system 100 presents the categories of data elements 112 to the controlling user 104, and the controlling user 104 selects the category of fear, leading to a subset of data elements 112 including, for example, data elements 112 corresponding to the user's fear of heights, such as hiking in the Grand Canyon and rock climbing, as well as the user's fear of spiders and bats.

The controlling user 104 may select a data element 112 related to user's fear of heights. The system 100 may be configured to present, to the controlling user 104, one or more additional data elements 112 associated with the same category as the selected data element, such as the user's fear of spiders. The controlling user 104 may then select the additional data element, fear of spiders, and the system 100 then presents a virtual reality representation of spiders to the participating user 102 in the virtual reality environment provided by the virtual reality device 106. In a further example, the psychotherapy VR system may present a series of photos to the participating user 102 and monitor the user's physiological responses to each photo to identify the most meaningful images and otherwise tailor the progress.

In some embodiments, an artist, such as an artificial intelligence artist, designs the virtual scene of the virtual environment 105 in which the session occurs. The virtual scene may be based on all or on a subset of the details of the personal database. In other embodiments, the virtual environment is photorealistic. In further embodiments, the virtual environment is animated.

As mentioned above, aspects of the systems and methods described herein are controlled by one or more controllers or processors. The one or more processors may be adapted to run a variety of application programs, access and store data, including accessing and storing data in the associated databases, and enable one or more interactions as described herein, including the execution of the methods and other features described above. Typically, the processor is implemented by one or more programmable data processing devices. The hardware elements, operating systems, and programming languages of such devices are conventional in nature, and it is presumed that those skilled in the art are adequately familiar therewith.

It should be noted that various changes and modifications to the presently preferred embodiments described herein will be apparent to those skilled in the art. Such changes and modifications may be made without departing from the spirit and scope of the present invention and without diminishing its attendant advantages.

We claim:

1. A psychotherapy virtual reality system configured to be used in the context of a psychotherapy session by a controlling user and a participating user, the psychotherapy virtual reality system comprising:
a virtual reality device configured to provide a virtual reality environment;
a database including one or more data elements derived from information associated with the participating user and provided by one of the controlling user and the participating user, wherein each data element is representative of the information;
a processor in communication with the virtual reality device and the database;
a memory in communication with the processor, the memory storing program instructions that, when executed by the processor, cause the processor to, in a psychotherapy session:
select, by the controlling user, a data element from the database to be used to construct a scenario in the virtual reality environment;
present, in the virtual reality environment provided by the virtual reality device, the scenario including a virtual reality representation of the selected data element to the participating user; and
control, by the controlling user, the virtual reality representation of the selected data element.

2. The psychotherapy virtual reality system of claim 1, wherein each data element comprises one of a person, an event, an activity, a series of events, a location, a place, a sound, a scent, or a word or phrase.

3. The psychotherapy virtual reality system of claim 2, wherein the scenario includes a plurality of virtual reality representations, wherein the data elements include a person, a place, and a phrase.

4. The psychotherapy virtual reality system of claim 2, wherein the processor is further configured to:
construct a dataset of the one or more data elements based on the information; and
store, via the database, the dataset.

5. The psychotherapy virtual reality system of claim 4, wherein the step of constructing the dataset comprises identifying, by the controlling user, an element disclosed during a conversation between the controlling user and the participating user, wherein each identified element corresponds to a data element.

6. The psychotherapy virtual reality system of claim 4, wherein the step of constructing the dataset comprises inputting one or more elements associated with the participating user, wherein each inputted element corresponds to a data element.

7. The psychotherapy virtual reality system of claim 1, wherein the database may further include information derived from one or more external data sources.

8. The psychotherapy virtual reality system of claim 7, wherein the one or more external data source comprises satellite imagery from transportation departments.

9. The psychotherapy virtual reality system of claim 1, wherein the processor is further configured to capture responsive data related to the participating user.

10. The psychotherapy virtual reality system of claim 9, wherein the responsive data comprises physiological data.

11. The psychotherapy virtual reality system of claim 10, further comprising one or more biological sensors configured to be worn by the participating user, wherein the step of capturing the responsive data comprises receiving, from the biological sensors, the physiological data.

12. The psychotherapy virtual reality system of claim 9, wherein the processor is configured to:
repeatedly present the virtual reality representation of the selected data element to the participating user during a plurality of psychotherapy sessions;
repeatedly capture the responsive data during the plurality of psychotherapy sessions; and
determine a data trend based on the captured responsive data.

13. The psychotherapy virtual reality system of claim 12, wherein the data trend indicates an improvement, no change, or a decline.

14. The psychotherapy virtual reality system of claim 1, wherein each data element is associated with a category.

15. The psychotherapy virtual reality system of claim 14, wherein each data element is associated into one or more of the following categories: anger, disgust, sadness, fear, shame, joy, pride, anxiety, excitement, or anticipation.

16. The psychotherapy virtual reality system of claim 14, wherein the processor is further configured to:
present, to the controlling user, one or more additional data elements, each additional data element being associated with the same category as the selected data element;
select, by the controlling user, an additional data element from the one or more additional data elements; and
present, in the virtual reality environment provided by the virtual reality device, a virtual reality representation of the selected additional data element to the participating user.
